(19) United States
(12) Patent Application Publication
(10) Pub. No.: US 2026/0218978 A1
(43) Pub. Date: Jul. 30, 2026

(54) IMAGER APPARATUS AND PROCESS FOR INGESTIBLE PRODUCTS

(71) Applicant: Messer Industries USA, Inc., Bridgewater, NJ (US)

(72) Inventor: John Griffiths, Shaker Heights, OH (US)

(73) Assignee: Messer Industries USA, Inc., Bridgewater, NJ (US)

(21) Appl. No.: 19/039,859

(22) Filed: Jan. 29, 2025

(51) Int. Cl. F25D 29/00; G01N 22/04; G01N 33/02; G01N 33/15

(52) U.S. Cl. CPC F25D 29/00; G01N 22/04; G01N 33/02; G01N 33/15

(57) ABSTRACT

An apparatus for providing a congelation profile of an ingestible product removed from a freezer, includes a transmitter for transmitting EMF radiation to contact the ingestible product; at least one sensor arranged at a side of the ingestible product for receiving the EMF radiation, the at least one sensor adapted to generate at least one signal responsive to the EMF radiation and representative of a dielectric of frozen portions of the ingestible product, and another dielectric of unfrozen portions of the ingestible product; and a controller in communication with the at least one sensor for receiving the at least one signal for producing therefrom the congelation profile of the frozen portions and the unfrozen portions of the ingestible product. A related process is also provided.

### BACKGROUND OF THE INVENTION

The present embodiments relate to apparatus and methods that sense and profile chilling and freezing characteristics and render related images of ingestible products such as for example food products and pharmaceuticals.

During known industrial processing of ingestible products such as for example U.S. FDA (Food and Drug Administration) regulated food products, and from a customer's perspective, effective chilling and freezing of a food product is the removal of an intended, specific amount of heat from the product. This heat removal is generally measured in BTU/lb (British Thermal Units per pound). For example, in order to calculate the specific BTUs actually removed from the food product, a technician at a plant or other processing site would take a physical sample of the product at the beginning of a freezing process, usually at an entrance or inlet of a freezer; followed by the taking of a physical sample of the product at an outlet end of the freezer. Using calorimetry, the technician would measure the amount of BTU/lb which have been removed from the food product after same transited the inlet through to the outlet. The technician would then know if the freezer and related freezing process for the food product is “tuned” correctly and efficiently for the particular type of food product being chilled or frozen. That is, the technician determines if the freezing process being employed is using a correct amount of freezing medium, and no more, to chill and/or freeze the food product so that the product is at the correct temperature when it emerges at the outlet of the freezer for subsequent processing. This known process, while accurate for calculating heat removal, is labor intensive and consumes time for the sampling, destroys some of the product during the sampling, and does not provide insight into the way in which the chilling and freezing has been performed upon the product.

Continuing with food products as an example, different food products cool and freeze differently. However, but all food products cool and freeze beginning from an exterior of the product toward an interior of the product. Therefore, depending upon the product's ability to conduct heat and the aggressiveness of the cooling or freezing process, two products with identical BTU/lb losses can exhibit or present different thermal profiles. For example, in a spiral freezer, the cooling process is generally slow and therefore, heat will be conducted from a center or core of the food product outward, resulting in a relatively even temperature profile (from center to the exterior) throughout the product. In contrast, a high heat transfer coefficient freezer provides a rate of cooling at higher orders of magnitude. Cooling and freezing of a food product in this type of high heat transfer coefficient freezer will create large temperature gradients throughout the product, potentially resulting in an uneven temperature profile of the product, i.e., the core of the food product being unfrozen while an exterior of the product is excessively chilled or perhaps frozen or perhaps exhibiting a frozen crust. Both products may exhibit identical heat removal in the calorimetry test, but in the former process for the product heat has been removed more uniformly and evenly.

A desirable result from freezing a food product is generally one wherein the core of the product does not exceed (is therefore at or below) a freezing point for the food product. Known methods to produce this desirable result include observing the thermal gradients in a product to determine if the core of the product is at or below the freezing point. These known methods also include inserting a thermocouple at specific intervals into the product as same is transported through the freezer, or dissecting the product and performing testing of same to determine the thermal gradient. Unfortunately, these known methods are destructive to the product, introduce heat thereby reducing accuracy of the thermal gradient measurements, can be done only on a periodic basis, and unnecessarily compromise efficiencies at the food production facility.

Therefore, it would be desirable and advantageous to have an apparatus and a process which provide an accurate thermal profile of an ingestible food or pharmaceutical product so that same is consistent throughout its chilling or freezing process without having to resort to known destructive processes to do so. It would also be desirable to have an apparatus and a process which provide a real-time signal about the thermal profile of the product so that the freezer operation can be adjusted to employ chilling and freezing changes to the food product being processed.

### SUMMARY OF THE INVENTION

There is therefore provided herein an apparatus for providing a congelation profile of an ingestible product removed from a freezer, which includes a transmitter for transmitting EMF radiation to contact the ingestible product; at least one sensor arranged at a side of the ingestible product for receiving the EMF radiation, the at least one sensor adapted to generate at least one signal responsive to the EMF radiation and representative of a dielectric of frozen portions of the ingestible product, and another dielectric of unfrozen portions of the ingestible product; and a controller in communication with the at least one sensor for receiving the at least one signal for producing therefrom the congelation profile of the frozen portions and the unfrozen portions of the ingestible product.

There is also provided herein a process for providing a congelation profile of an ingestible product removed from a freezer, which includes transmitting EMF radiation for contacting the ingestible product; sensing the EMF radiation at the ingestible product with at least one sensor for generating at least one signal responding to the EMF radiation and representing a dielectric of frozen portions of the ingestible product, and another dielectric of unfrozen portions of the ingestible product; and providing a controller for receiving the at least one signal for producing therefrom the congelation profile of the frozen portions and the unfrozen portions of the ingestible product.

### BRIEF DESCRIPTION OF THE DRAWINGS

For a more complete understanding of the present invention, reference may be had to the following description of exemplary embodiments considered in connection with the accompanying drawing Figure(s), of which:

FIG. 1 shows a schematic of a first embodiment of an apparatus and a process according to the present invention.

FIG. 2 shows a schematic of another view of the embodiments shown in FIG. 1.

FIGS. 3A-3C show schematic views of another embodiment of an apparatus and a process according to the present invention.

FIGS. 4A-4B show schematic views of still another embodiment of an apparatus and a process according to the present invention.

FIGS. 5A-5B show schematic views of still another embodiment of an apparatus and a process according to the present invention.

### DETAILED DESCRIPTION OF THE INVENTION

Before explaining the inventive embodiments in detail, it is to be understood that the invention is not limited in its application to the details of construction and arrangement of parts illustrated in the accompanying drawings, if any, since the invention is capable of other embodiments and being practiced or carried out in various ways. Also, it is to be understood that the phraseology or terminology employed herein is for the purpose of description and not of limitation.

In the following description, terms such as for example horizontal, upright, vertical, above, below, beneath and the like, are to be used solely for the purpose of clarity illustrating the invention and should not be taken as words of limitation. The drawings are for the purpose of illustrating the invention and are not intended to be to scale. A dielectric of a product changes significantly when the product freezes. Use of the term “thermal” herein pertains to of, relating to, or caused by heat, and being or involving a state of matter dependent upon temperature. Therefore, use of the term “thermal profile” or “profile” herein pertains to a temperature aspect of a product, that fraction or portion of a product which is frozen, and that fraction or portion of a product which is unfrozen. Use of the term “congelation” as a noun pertains to a process or a result of “to congeal” which is to change from a fluid to a solid state by or as if by cold.

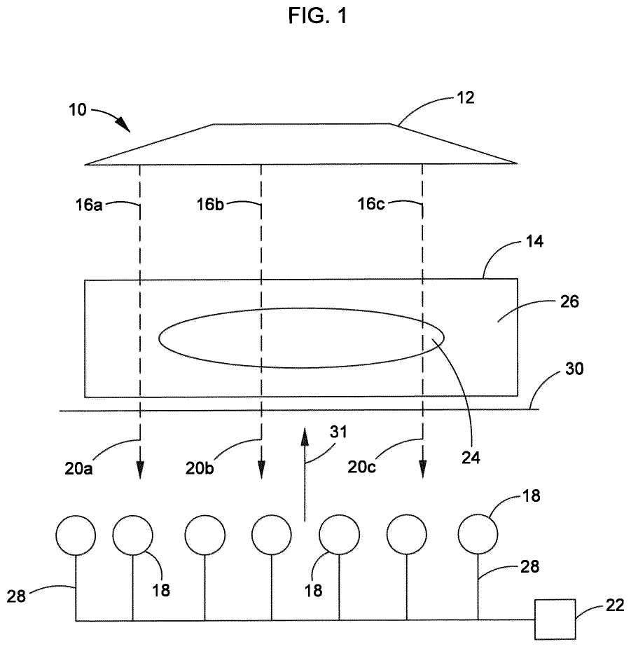

The apparatus and process embodiments described herein and shown in FIGS. 1-2, 3A-3C, 4A-4B and 5A-5B use a difference, delta or a change that occurs to a property of an ingestible product when such is chilled and/or frozen. The ingestible product may be an ingestible food product for humans and other animals, and may be an ingestible pharmaceutical product for humans and other animals. In many instances, the product, whether food or pharmaceutical, has just completed being chilled or frozen. For the sake of brevity and uniformity, reference herein to the “food” or “product” also includes an ingestible food product for humans and other animals, such as by a dog or a cat for example, and an ingestible pharmaceutical product for humans and other animals, such as a dog or a cat for example.

More particularly, the inventive embodiments provide insight to and utilize the change that occurs to the dielectric property (a/k/a the “dielectric”) of any water (regardless of phase) present within the product as a temperature of the product is reduced during chilling and freezing.

The dielectric property of a product is the way in which a medium (a solid, liquid or gas) of the product responds to the presence of electromagnetic waves or an electromagnetic field (individually and collectively referred to herein as “EMF”). The medium may be the actual product, or the solid, liquid and/or gas that is included in the product. The dielectric property of the medium is an electromagnetic repulsive property of the medium, and the repulsive property is opposite to that which occurs during conduction. In conduction, electrons move or are conducted through (not repulsed by) the medium. In contrast, the dielectric of a medium is the molecules of the medium pushing back, repulsing or exerting a force against the EMF.

Therefore, a medium with a high dielectric property or value has molecules which are highly sensitive to the electromagnetic waves, or EMF, and accordingly, the molecules are able to reorientate themselves with little energy within the medium. Water for example has a high dielectric property (a high dielectric property number) and therefore, the water molecules have high mobility, are able to repulse the electromagnetic waves which results in reduced transparency of the water to the EMF. Water in a liquid state or phase typically has a dielectric value of 80. By comparison, air in a gaseous state or phase (as opposed to liquid air) has a low dielectric value of 1. In a capacitor for example, materials with high dielectrics are used in the capacitor to thereby push back against or repulse the EMF which increases the working capacity of the capacitor element.

A medium with a low dielectric property or value (such as air) has molecules within the medium which are substantially, if not already, immobilized such that the molecules are not able to reorientate themselves within the medium. In addition, materials having a medium which does not interact with electromagnetic radiation at a specific frequency will also exhibit a low dielectric property. Frozen water for example has a low dielectric property (a low dielectric property number) and therefore, the frozen water molecules have reduced to no mobility. The molecules are such as if frozen in crystalline structure and accordingly, are unable to reorientate and repulse the EMF, which results in the medium being substantially transparent to the EMF. Frozen water or ice typically has a dielectric value of 3. By comparison, air has a dielectric value of 1. In a wire for example, materials with low dielectrics are used so that the magnetic field can leak out of the wire, while a resistance of the wire remains low.

The present embodiments use the dielectric property effect. To put this in context with respect to a food, as any water within the food cools, its dielectric or rather the dielectric of the water content in the food changes. In other words, as the water in the food cools, a dielectric value of the water decreases, i.e., becomes lower. A dielectric of any fats or proteins in the food remains substantially constant.

In contrast, and as explained above, molecules in a warm water medium are very mobile and therefore, the molecules can easily self-orientate when exposed to an electromagnetic field or EMF. As a result, warm water molecules tend to have high dielectric values. However, in ice, the frozen water is locked into a crystalline structure resulting in a reduced mobility for the water molecules and therefore, the ice has a low dielectric value.

The dielectric value of a food product above its freezing point will have a gradual reduction of its dielectric value as the food product is cooled. Because food products in particular contain water soluble proteins and other substances, the existing water does not freeze uniformly in the food product when same is exposed to a single temperature. A pharmaceutical may have similar characteristics regarding its constituents. That is, the different constituents of the pharmaceutical may have different dielectrics. Typically, the food product will freeze over a range in temperature of about 10° F. Once the food reaches the start of its freezing point, a portion of the water in the food becomes immobilized as ice crystals, resulting in a reduction in the dielectric of the more frozen portion of the food product and a reduction in the overall dielectric of the food product. Other portions of the food may continue to have liquid water present within cells of the food product and therefore, the dielectric property remains higher in the food product at those portions containing liquid water. As the food product cools further toward freezing, an increased fraction or proportion of the water molecules becomes locked into ice crystals, thereby further reducing the dielectric of the food product. And finally, once the entire food product is completely frozen, the water molecules become crystallized molecules which are relatively passive to the incoming electromagnetic waves, wherein the food product is effectively rendered transparent to the microwaves.

During the above process of chilling and the subsequent freezing, a thermal profile of the food product changes. The thermal profile can be sensed and accurately represented to enable a food processor to understand the changing thermal profile of the food product and to what extent, if any, the freezing process is to be adjusted for the food product.

Two methods are generally used to measure the dielectric of an item, composition or substance (for the sake of brevity referred to herein individually and collectively as an item or a medium). Both methods involve directing an electromagnetic wave or an EMF toward the medium in an attempt to pass the wave through the medium.

In the first method, similar to that used by the U.S. Transportation Security Administration (TSA) in TSA scanners, microwaves are directed to contact and move through a person to discover object(s) on the person. Based upon the dielectric of the medium encountered by the microwaves, i.e., the fabric, flesh, and other materials present on the person, the amount of microwaves which pass into or are repelled by the person (the “medium”) will change. For example, metals (a metallic medium), having a higher dielectric property and therefore a lower transparency, on the person will tend to repel the microwaves, resulting in an increase in the amount of microwaves reflected back to a source of the microwave transmission (the microwave transmitter). In conjunction, a back-scattering scanner receiving the repelled microwaves is used to sense or see the dielectric of a surface of the medium and the dielectric for any item that is within the medium. Accordingly, a signal is generated and visually displayed representing what has been sensed on the person.

The second method is used to measure an amount of electromagnetic radiation that passes or is transmitted through the medium. An area of a medium with a high dielectric property will tend to prevent or be opaque to this transmission, while an area of the medium having a low dielectric property will tend to be substantially if not entirely transparent to the transmission. This explains why frozen products, such as frozen food products, having a low dielectric, require increased time to be defrosted in the microwave. The frozen food with the low dielectric is relatively transparent to the microwaves, thereby resulting in the microwaves being transmitted through the frozen food medium without actuating the heating effect of the waves within the frozen food. This is in contrast to ambient temperature food products, i.e., products above the freezing point, having a higher dielectric which causes the products to be opaque to the microwaves. Microwaves thereby interact with the food product, resulting in energy transfer to the dielectric molecules. This results in heating of the product, i.e., not all of the energy transmitted to the product is reflected back or away from the product. Therefore, the dielectric of the material of the product facilitates energy transfer from the microwaves to the product and thereby causes heating of the product.

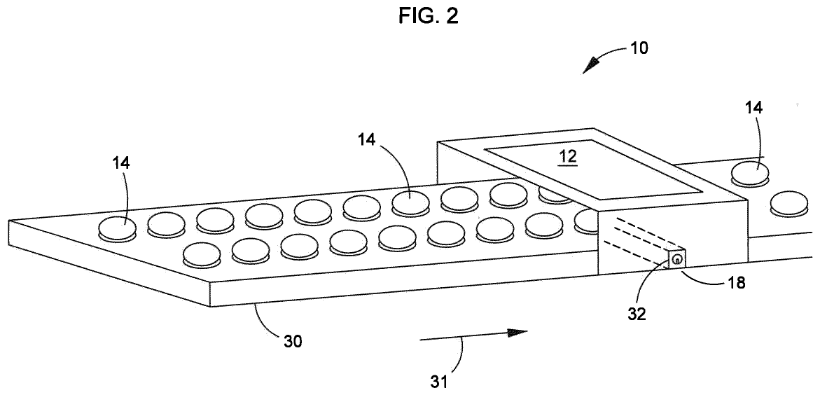

Referring to FIGS. 1 and 2, the present embodiments include an apparatus shown generally at 10 and a low power electromagnetic source (e.g., a microwave transmitter) 12 supported above or at one side of a food stuff 14, food product, pharmaceutical or pharmaceutical product (hereinafter the “product” or “products”). The transmitter 12 is positioned downstream from a freezer (not shown), and measures an amount of EMF radiation or myriad of waves 16a-16c directed at and to pass through the product 14. A conveyor belt 30 transports the product 14 in the direction of arrow 31 from the freezer to beneath the transmitter 12. Beneath the product 14 and the conveyor 30 is arranged at least one and for most applications a plurality (such as an array) of sensors 18. The sensors 18 are fixed in place and may be mounted in a region 32 or trough which spans an underside of the belt 30 as shown in FIG. 2. The belt 30 may be fabricated from high density polyethylene or other similar material having a low dielectric property value. That is, the belt 30 is constructed of a material which is transparent to the electromagnetic waves (the microwaves) 16a-16c.

The sensors 18 each receive a myriad of transmitted electromagnetic waves 20a,20b,20c that have passed in some amount through the product 14 and the conveyor belt 30 in a manner that is representative of the dielectric characteristic of that portion or region of the product. It is understood that the waves 20a-20c shown in FIG. 1 are by way of example only for the sake of brevity and to illustrate what is occurring in this embodiment, while during operations the actual waves 16a-16c are transmitted continuously in an amount large enough to cover or blanket that area or region of the product 14 to be sensed.

The intensity of the waves 20a-20c received by the sensors 18 are converted into signals 28 transmitted to a controller 22, wherein a value is allocated to each of the signals. The higher the value of a signal's intensity, the greater the intensity of the electromagnetic waves that have passed or been transmitted through the product 14. Conversely, the lower an intensity of the signal of the electromagnetic waves that have passed or been transmitted through the product 14, such is representative of a greater portion of the waves having been reflected back or repulsed towards the transmitter 12. Referring to FIG. 1, the electromagnetic wave 20a would be representative of a high signal resulting from the wave contacting a frozen portion 26 of the product 14; the electromagnetic wave 20b would be representative of a low signal resulting from the wave contacting an unfrozen portion 24 of the product such as unfrozen water in the product; and the electromagnetic wave 20c would be representative of a medium signal resulting from the wave contacting a partially frozen water portion of the product, i.e., neither frozen nor completely unfrozen. Where there are areas of unfrozen 24 or a non-solid medium in the product 14, such as partially frozen water, as opposed to a frozen portion 26 of the product, the intensity of a signal 28 from each of the sensors 18 would be reduced as the electromagnetic waves are reflected back toward the transmitter 12 or adsorbed by the product 14 due to the higher dielectric of the unfrozen portion 24. The lower strength signal transmitted through the product 14 results from the higher amount of energy reflected back to the transmitter 12. This lower received signal would therefore correlate to an amount of freezing that has taken place inside the product 14. As shown in FIG. 1, a high signal 20a is representative of an area of the product 14 that is frozen due to the increased intensity of the signal, while a low signal 20b is representative of a lower intensity signal due to the higher dielectric from the unfrozen water containing portion of the product 14 reflecting the signal 16b back to the transmitter 12. In this embodiment, the sensing is with respect to what actually passes through the product 14 to the sensors 18, not what is reflected back to the transmitter 12. In effect, any reflected signal would be represented as a drop in the received signal, i.e., the product 14 does not generate the signal but rather, it either transmits or reflects the majority of the signal. The signal 20c is of medium strength, compared to the signals 20a and 20b, as this signal has been transmitted through the portion 24 of the product 14 containing unfrozen water at this area of the product and accordingly, a lesser amount of the signal 20c is reflected back to the transmitter 12 while a greater portion of the signal passes through the product to be received by the sensor 18.

Because the product 14 is supported upon and moved by the conveyor

belt 30 constructed from a low dielectric plastic, such as for example a low dielectric plastic mesh conveyor belt, the sensors 18 and transmitter 12 can be arranged in a row. The motion of the product 14 supported and carried by the conveyor belt 30 over the sensors 18 will generate a signal representative of a heat signature profile for the product, i.e., how much of the product is frozen and where the frozen portions are located in the product. For example, the apparatus embodiment of FIGS. 1 and 2 can include the conveyor belt 30 having a 48-inch width for transporting the products 14, and a plurality of the sensors 18 (for example, forty-eight sensors) in the row. As the product 14 is moved by the conveyor belt 30, the movement of the product contacted by the waves 16a-16c will provide a profile from the signals 20a-20c of the internal dielectric of the product. Therefore, instead of a digital camera, which needs a grid of sensors, the present embodiments can merely use a row of the sensors 18 (at much lower cost), as such would use movement of the product 14 to generate an image of what is frozen versus what remains unfrozen within the product, i.e., the thermal profile of the product. The controller 22 can therefore subsequently display the congelation profile of the product, from which the operator can adjust the amount of chilling or freezing to be applied to the product in the freezer.

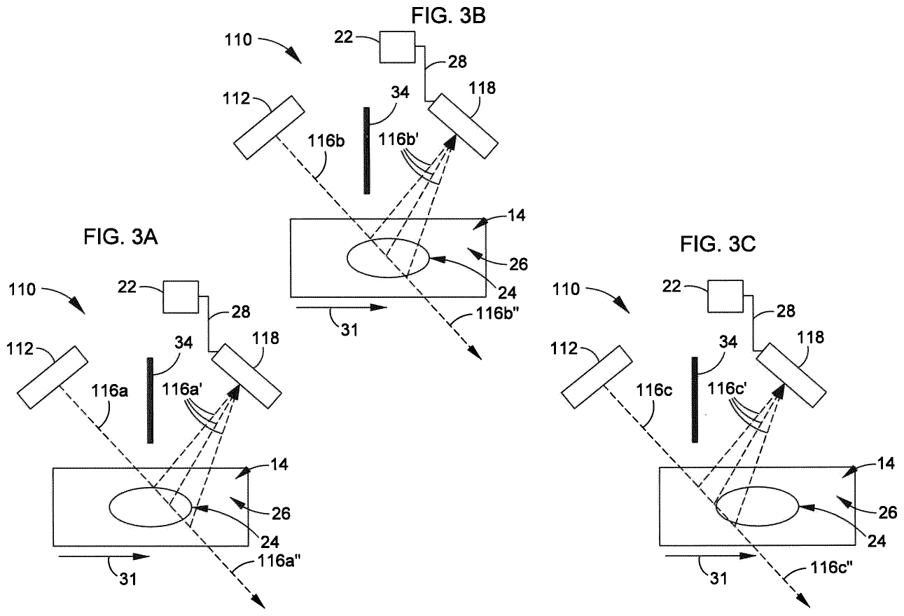

Referring to the views of FIGS. 3-5, the use of shorter wavelengths of radiation with higher frequency results in a higher resolution of an image and a congelation profile of the product 14. The frequency used correlates to the ability to visualize an object. That is, the relationship between frequency and wavelength for EMF is given by the equation λf=c; where λ is the wavelength, f is the frequency, and c is the speed of light. In addition, it is typical for the minimum dimension which can be resolved to be approximately half of the wavelength. For example, the use of a frequency of 3 GHz is similar to the frequency used in a typical microwave oven (which operates at 2.45 GHz). This EMF has a wavelength of about 10 cm or 4 inches. Therefore, microwaves at a frequency of 3 GHz will be able to resolve objects no smaller than about 2 inches in diameter. In order to observe higher resolution of the object, higher frequencies would be required. Therefore, by way of example, a 60 GHz EMF has a wavelength of 5 mm, and such would be able to resolve unfrozen material up to 2.5 mm or 1/10 inch in diameter.

If the product 14 is too thick, a low power electromagnetic source may not generate waves that can sufficiently penetrate and pass through the product. This is because the sensor 118 would need to detect the difference between microwaves passing through the food product 14 (the “signal”) and the background noise resulting from reflections from nearby objects (the “noise”). The ratio of signal to noise (signal: noise) is critical to be able to measure what fraction of the radiation is reflected or repelled by the unfrozen food product. As a result, the low power electromagnetic source would have difficulty accurately imaging that portion of the product which is unfrozen. If the amount of power needed to create a significant signal to noise ratio is too high, there is the potential for safety and/or product heating issues. In this scenario, it would be more appropriate to use a back-scattering embodiment, wherein the sensors 118 and the transmitter 112 (the electromagnetic source) are on a same side of the product 14, as shown in FIGS. 3A-3C.

Referring now to the views of FIGS. 3A-3C, in these embodiments of the apparatus 110 the sensors 118 are fixed and can be arranged collectively in an array (the sensor array) which is positioned to capture the electromagnetic waves reflected off the product 14. The conveyor belt 30 moves in the direction of the related arrow 31 to deliver the products 14 to pass first by the transmitter 112. A barrier 34 or wall constructed from a high dielectric material, such as metal, is positioned between the transmitter 112 and the sensor array 118 to prevent waves generated from the transmitter inadvertently being directly received by the array. The barrier 34 prevents the sensor array 118 from observing high levels of electromagnetic fields (EMF) directly from the transmitter 112, which would drown-out the sensor array and prevent same from being able to observe or sense low intensity EMF (116a′-116c′) reflected from the product 14. In effect, the barrier 34 shields or protects the array 118 from receiving unnecessary levels of the EMF which could compromise an accurate presentation of the congelation profile of the product 14.

As shown in FIGS. 3A, 3B, 3C, a wave 116a from the transmitter 112 proceeds through the frozen portion 26 of the product 14 until it contacts the unfrozen liquid portion 24 at which point a portion of the wave 116a is reflected in a wave 116a′ toward the sensor array 118 as the wave 116a passes through the product 14. This reflection to provide the wave 116a′ does not take place at a single point within the product 14 but rather, it takes place continuously as 116a passes through the product 14. The remaining portion of the wave 116a which is not reflected, herein 116a″, exits the product 14 and is not detected by the receiver 118. A signal 28 from the waves 116a′ received by the array 118 is generated and delivered to the controller 22 to provide a visual representation of the waves reflected from the product 14 and received by the array, such as on a display screen, which could be brightly illuminated regions to indicate a larger portion of the wave 116a has been reflected as the wave 116a′ to the array. The display from the controller 22 in all embodiments can include lights, lighted bars, lighted bar graphs, and color combinations thereof, for example.

As shown in FIG. 3B, the product 14 continues to move from left to right with the conveyor belt 30 in the direction 31 as the waves 116b are continuously directed to the product. As the product 14 is moving, the amount of unfrozen portion 24 that is exposed to the waves is changing. When a greater portion of the product 14 is the unfrozen portion 24, a larger or greater amount of the wave 116b is reflected as a wave 116b′ to the sensor array 118 in view of the wave 116b becoming increasingly exposed to the unfrozen portion 24 of the product. This will result in a relatively high signal being received at the array 118, and a decreased wave 116b″ being transmitted through and exiting the product 14.

Referring now to FIG. 3C, as the product 14 departs from exposure to the sensor array 118, the product will have a greater portion which is completely frozen and exposed to the transmitter 112. This frozen portion 26 has a lower dielectric and therefore, will be less reflective to the EMF. As the volume of the unfrozen portion 24 begins to decrease, a reduced or lesser portion of the product 14 will be reflective to the wave 116c. As a result, the amount of the waves 116c′ reflected and thereafter received by the array 118 will decrease, while an amount of the waves 116c″ passing through the product 14 will increase. As a result, the sensor array 118 will receive a varying amount of reflected waves which correlates to the amount of unfrozen material present in the product 14. The controller 22 will receive the signals 28 of the waves 116a′; 116b′; 116c′ from the sensor array 118 and display a visual representation of the congelation profile of the product 14.

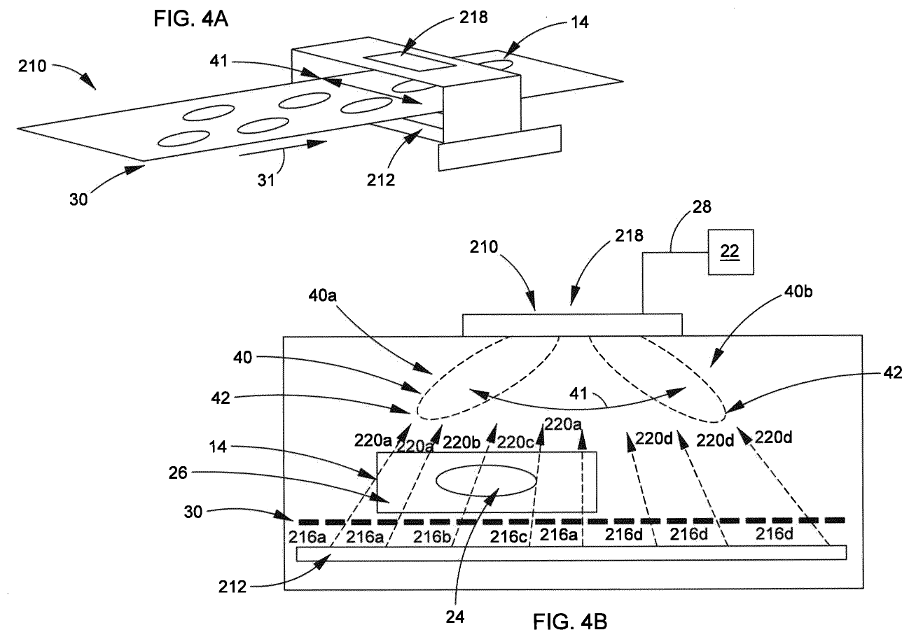

Referring now to FIGS. 4A, 4B, in these embodiments of the apparatus 210 the electromagnetic or phased array receiver 218 or sensor may be constructed as a phased array, wherein a plurality of receivers are shown arranged or mounted together as a single unit. The receiver 218 utilizes an interference pattern 40, wherein a sensitivity at the pattern is applicable to a narrow sensitivity window 42 (or “window”) of the available EMF waves provided for being received at the receiver. In contrast and by way of example, the embodiments shown in FIGS. 1 and 3A-3C include the sensors 18, 118 being fixed for receiving the waves from the passing products 14 on the conveyor belt 30. Therefore, in order to increase the number of points (or the products 14) sensed across the belt 30, the number of the sensors 218 in the embodiments of FIGS. 4A-4B must be increased over the number of sensors 18,118 used in the embodiments of FIGS. 1 and 3A-3C. Accordingly, the embodiments of FIGS. 4A-4B call for the use of the interference pattern 40 derived from the phased array 218 which enables a single sensor to be steered or directed from one side of the conveyor belt 30 to another side of the same conveyor belt. As a result, the phased array receiver 218 of FIGS. 4A-4B does not need to physically move or change position but rather, the phased array receiver will continue to function as though it consists of a plurality of separate sensors (receivers). In the phased array receiver 218, incoming signals or waves 220a, 220b, 220c, 220d (or 220a-220d) generated or provided from the electromagnetic source or transmitter 212 are cancelled out electronically so that only those signals which pass through the window 42 of the interference pattern 40 will remain. By changing which of the incoming signals 220a-220d are cancelled, it is possible to sweep (arrow 41) the window 42 from one side of the belt 30 (at location 40a) to the another side of the belt (at location 40b), repetitively, as shown in FIG. 4B. By recording the intensity of those signals 220a-220d of the EMF passing through the window 42 during the sweep 41, profile data can be collected from all points, or from all the products 14 across the belt 30. As a result, the phased array receiver 218, fixed in position, will function similar to a plurality of receivers covering a location spanning a width across the belt 30. In this embodiment of FIGS. 4A-4B, the EMF microwave source or transmitter 212 is usually fixed, while the sensor 218 or the receiver sweeps its sensitivity window 42 using the phased array across the conveyor belt 30. The waves 220a-220d in this embodiment are similarly exposed to the frozen portion 26 and the unfrozen portion 24 of the product 14 as same passes by and between the transmitter 212 and the phased array sensor 218 on the conveyor belt 30 so that a signal 28 generated by the sensor 218 can be transmitted to the controller 22. The controller 22 will generate a congelation profile of the product 14 for display.

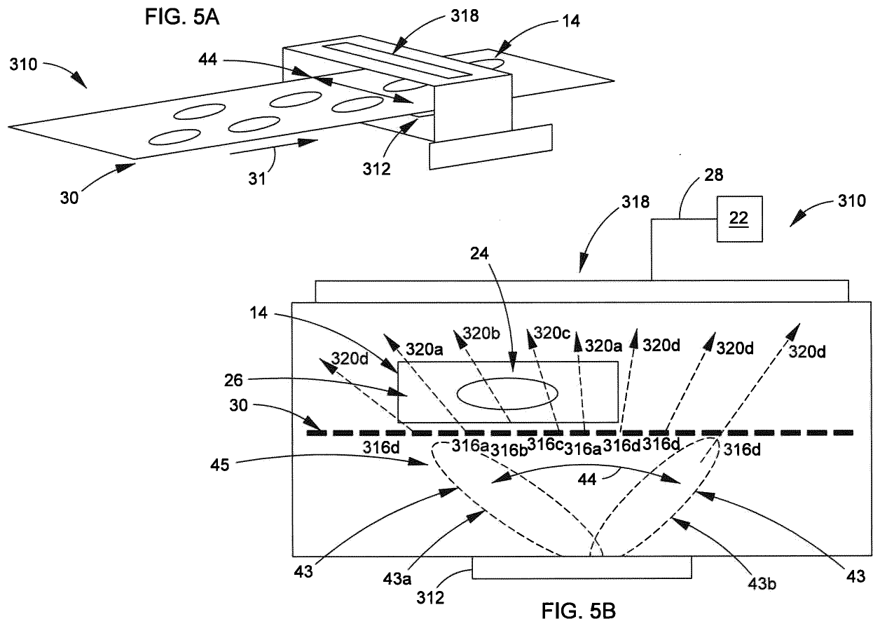

In the embodiments of FIGS. 5A-5B, there is provided an apparatus 310 including a single sensor 318 or receiver which observes or senses the product 14 on the conveyor belt 30, and an oscillating EMF beam 43 that is generated or provided from the transmitter 312 and is swept across the conveyor belt upon which the product is transported. In the embodiment shown in FIG. 5B, an EMF transmitter 312 is a phased array transmitter or phased array. The EMF beam 43 provides an interference pattern 45 as a narrow, focused beam of EMF transmitted towards the product 14 on the belt 30. The single stationary receiver 318 receives the EMF signals 320a, 320b, 320c, 320d depending upon how much of the EMF radiation passes through the product 14. By oscillating the interference pattern 43 generated from the EMF transmitter 312, the transmitter functions like a phased array transmitter able to sweep (arrow 44) the beam from one side 43a of the belt 30 to another side 43b of the belt, repetitively, as shown in FIG. 5B. The timing of the sampling of the signals 320a-320d received by the receiver 318 enables the combination of the transmitter 312 and the receiver 318 to coact with a functional equivalency to that of a plurality of receivers located for spanning the belt 30. The waves 320a-320d in this embodiment are similarly exposed to the frozen portion 26 and the unfrozen portion 24 of the product 14 as same passes by and between the transmitter 312 and the phased array sensor 318 on the conveyor belt 30 so that a signal 28 generated by the sensor 318 can be transmitted to the controller 22. The controller 22 will generate a congelation profile of the product 14 for display.

The present embodiments in FIGS. 1-2, 3A-3C, 4A-4B and 5A-5B do not cook or heat the product 14 and therefore, the microwaves will be safer than those waves generated in a home microwave oven. Further, the microwaves or beam can be contained in for example a container using a Faraday cage device (not shown) so that persons proximate the area of operations with the present embodiments are shielded and unaffected by the apparatus and process embodiments.

Examples of the chilled or frozen food products 14 include, but are not limited to, chicken breasts, chicken wings, beef burgers, pizzas, and bakery products.

Examples of the chilled or frozen pharmaceutical products 14 include, but are not limited to, vaccines, tissue samples, bulk drugs, and precursors.

There is provided herein a first apparatus embodiment for providing a congelation profile of an ingestible product removed from a freezer, comprising: a transmitter for transmitting EMF radiation to contact the ingestible product; at least one sensor arranged at a side of the ingestible product for receiving the EMF radiation, the at least one sensor adapted to generate at least one signal responsive to the EMF radiation and representative of a dielectric of frozen portions of the ingestible product, and another dielectric of unfrozen portions of the ingestible product; and a controller in communication with the at least one sensor for receiving the at least one signal for producing therefrom the congelation profile of the frozen portions and the unfrozen portions of the ingestible product.

A second apparatus embodiment includes the first apparatus embodiment, wherein the transmitter and the at least one sensor are arranged at different sides of the ingestible product.

A third apparatus embodiment includes the first apparatus embodiment, wherein the transmitter is arranged at a side of the ingestible product proximate the at least one sensor.

A fourth apparatus embodiment includes the third apparatus embodiment, wherein the transmitter and the at least one sensor are constructed as an integral unit.

A fifth apparatus embodiment includes the fourth apparatus embodiment, wherein the at least one sensor is constructed and arranged as a sensor array.

A sixth apparatus embodiment includes the fifth apparatus embodiment, wherein the transmitter is constructed and arranged to steer the EMF radiation to sweep across the ingestible product in a plurality of repetitive motions to generate the at last one signal.

A seventh apparatus embodiment includes the fifth apparatus embodiment, wherein the at least one sensor is constructed and arranged to steer radiation sensitivity of the EMF radiation to sweep across the ingestible product in a plurality of repetitive motions to generate the at last one signal.

An eighth apparatus embodiment includes the fifth apparatus embodiment, wherein the transmitter and the sensor array are each constructed and arranged to sweep across the ingestible product in a plurality of repetitive motions, wherein the transmitter provides an interference pattern of the EMF radiation toward the ingestible product, and the at least one sensor steers radiation sensitivity of the EMF radiation from the ingestible product to the sensor array.

A ninth apparatus embodiment includes the first apparatus embodiment, wherein the controller is adapted to adjust operation of the freezer for which the apparatus is associated in response to the at least one signal received from the at least one sensor.

A tenth apparatus embodiment includes the first apparatus embodiment, wherein the ingestible product is selected from the group consisting of a food product, and a pharmaceutical product.

An eleventh apparatus embodiment includes the first apparatus embodiment, wherein the congelation profile of the ingestible product is represented by visual representations selected form the group consisting of lights, lighted bars, lighted bar graphs, and color combinations thereof.

There is provided herein a first process embodiment for providing a congelation profile of an ingestible product removed from a freezer, comprising: transmitting EMF radiation for contacting the ingestible product; sensing the EMF radiation at the ingestible product with at least one sensor for generating at least one signal responding to the EMF radiation and representing a dielectric of frozen portions of the ingestible product, and another dielectric of unfrozen portions of the ingestible product; and providing a controller for receiving the at least one signal for producing therefrom the congelation profile of the frozen portions and the unfrozen portions of the ingestible product.

A second process embodiment includes the first process embodiment, further comprising arranging the transmitter and the at least one sensor at different sides of the ingestible product.

A third process embodiment includes the first process embodiment, further comprising arranging the at least one sensor at a side of the ingestible product proximate the EMF radiation.

A fourth process embodiment includes the first process embodiment, further comprising sweeping the EMF radiation across the ingestible product in at least one or a plurality of repetitive motions for generating the at least one signal.

A fifth process embodiment includes the first process embodiment, further comprising steering radiation sensitivity of the EMF radiation to sweep across the ingestible product in at least one motion or a plurality of repetitive motions for generating the at least one signal.

A sixth process embodiment includes the first process embodiment, wherein the transmitting the EMF radiation and the sensing the EMF radiation are at an integral unit functioning as a sensor array, and further comprising sweeping the EMF radiation repetitively across the ingestible product for generating the at least one signal representing the congelation profile of the ingestible product.

A seventh process embodiment includes the sixth process embodiment, further comprising providing the at least one signal as a visual display of the congelation profile for the ingestible product.

An eighth process embodiment includes the first process embodiment, further comprising adjusting operation of the freezer in response to the at least one signal sensed.

A ninth process embodiment includes the first process embodiment, wherein the ingestible product is selected from the group consisting of a food product, and a pharmaceutical product.

It will be understood that the embodiments described herein are merely exemplary, and that a person skilled in the art may make variations and modifications without departing from the spirit and scope of the invention. All such variations and modifications are intended to be included within the scope of the invention as described above and provided for in the appended claims. It should be understood that the embodiments described above are not only in the alternative but can be combined.

What is claimed is:

1. An apparatus for providing a congelation profile of an ingestible product removed from a freezer, comprising:
a transmitter for transmitting EMF radiation to contact the ingestible product;
at least one sensor arranged at a side of the ingestible product for receiving the EMF radiation, the at least one sensor adapted to generate at least one signal responsive to the EMF radiation and representative of a dielectric of frozen portions of the ingestible product, and another dielectric of unfrozen portions of the ingestible product; and
a controller in communication with the at least one sensor for receiving the at least one signal for producing therefrom the congelation profile of the frozen portions and the unfrozen portions of the ingestible product.

2. The apparatus of claim 1, wherein the transmitter and the at least one sensor are arranged at different sides of the ingestible product.

3. The apparatus of claim 1, wherein the transmitter is arranged at a side of the ingestible product proximate the at least one sensor.

4. The apparatus of claim 3, wherein the transmitter and the at least one sensor are constructed as an integral unit.

5. The apparatus of claim 4, wherein the at least one sensor is constructed and arranged as a sensor array.

6. The apparatus of claim 5, wherein the transmitter is constructed and arranged to steer the EMF radiation to sweep across the ingestible product in a plurality of repetitive motions to generate the at last one signal.

7. The apparatus of claim 5, wherein the at least one sensor is constructed and arranged to steer radiation sensitivity of the EMF radiation to sweep across the ingestible product in a plurality of repetitive motions to generate the at last one signal.

8. The apparatus of claim 5, wherein the transmitter and the sensor array are each constructed and arranged to sweep across the ingestible product in a plurality of repetitive motions, wherein the transmitter provides an interference pattern of the EMF radiation toward the ingestible product, and the at least one sensor steers radiation sensitivity of the EMF radiation from the ingestible product to the sensor array.

9. The apparatus of claim 1, wherein the controller is adapted to adjust operation of the freezer for which the apparatus is associated in response to the at least one signal received from the at least one sensor.

10. The apparatus of claim 1, wherein the ingestible product is selected from the group consisting of a food product, and a pharmaceutical product.

11. The apparatus of claim 1, wherein the congelation profile of the ingestible product is represented by visual representations selected form the group consisting of lights, lighted bars, lighted bar graphs, and color combinations thereof.

12. A process for providing a congelation profile of an ingestible product removed from a freezer, comprising:
transmitting EMF radiation for contacting the ingestible product;
sensing the EMF radiation at the ingestible product with at least one sensor for generating at least one signal responding to the EMF radiation and representing a dielectric of frozen portions of the ingestible product, and another dielectric of unfrozen portions of the ingestible product; and
providing a controller for receiving the at least one signal for producing therefrom the congelation profile of the frozen portions and the unfrozen portions of the ingestible product.

13. The process of claim 12, further comprising arranging the transmitter and the at least one sensor at different sides of the ingestible product.

14. The process of claim 12, further comprising arranging the at least one sensor at a side of the ingestible product proximate the EMF radiation.

15. The process of claim 12, further comprising sweeping the EMF radiation across the ingestible product in at least one or a plurality of repetitive motions for generating the at least one signal.

16. The process of claim 12, further comprising steering radiation sensitivity of the EMF radiation to sweep across the ingestible product in at least one motion or a plurality of repetitive motions for generating the at least one signal.

17. The process of claim 12, wherein the transmitting the EMF radiation and the sensing the EMF radiation are at an integral unit functioning as a sensor array, and further comprising sweeping the EMF radiation repetitively across the ingestible product for generating the at least one signal representing the congelation profile of the ingestible product.

18. The process of claim 17, further comprising providing the at least one signal as a visual display of the congelation profile for the ingestible product.

19. The process of claim 12, further comprising adjusting operation of the freezer in response to the at least one signal sensed.

20. The process of claim 12, wherein the ingestible product is selected from the group consisting of a food product, and a pharmaceutical product.
